(19) United States
(12) Patent Application Publication
(10) Pub. No.: US 2003/0113322 A1
(43) Pub. Date: Jun. 19, 2003

(54) CDR-H1-derived peptide CB11, pharmaceutical compositions made therefrom and methods of treating disorders in mammals

(76) Inventors: Cedric Bes, Montpellier (FR); Laurence Briant-Longuet, Gallargues (FR); Martine Cerutti, Saint-Christol-Lez-Ales (FR); Thierry Chardes, Assas (FR); Gerard DeVauchelle, Saint-Christol-Les-Ales (FR); Christian DeVaux, Montpellier (FR); Claude Granier, Clapiers (FR); Claude Mawas, Marseille (FR); Daniel Olive, Marseille (FR); Bernard Pau, Montpellier (FR)

Correspondence Address: IP DEPARTMENT OF PIPER RUDNICK LLP, 3400 TWO LOGAN SQUARE, 18TH AND ARCH STREETS, PHILADELPHIA, PA, 19103, US

(21) Appl. No.: 10/322,142

(22) Filed: Dec. 18, 2002

Related U.S. Application Data
(60) Provisional application No. 60/374,754, filed on Apr. 23, 2002.
(60) Provisional application No. 60/341,349, filed on Dec. 18, 2001.

(51) Int. Cl. A61K 39/395; C07K 16/28

(52) U.S. Cl. 424/144.1; 530/388.22

(57) ABSTRACT

An isolated CDR-derived polypeptide from monoclonal antibody 13B8.2, a pharmaceutical composition including a pharmaceutically acceptable carrier and at least a peptide, and a method for treating a subject suffering from an autoimmune disorder, including administering to the subject a therapeutically effective amount of a pharmaceutical composition, a method for treating a subject suffering from a transplant rejection including administering to the subject a therapeutically effective amount of a pharmaceutical composition, a method for treating a subject suffering from an HIV immunodeficiency disorder including administering to the subject a therapeutically effective amount of a pharmaceutical composition and a method for treating a subject suffering from a tumoral disorder including administering to the subject a therapeutically effective amount of a pharmaceutical composition.

### RELATED APPLICATIONS

This application claims priority of U.S. Provisional Application Nos. 60/341,349, filed Dec. 18, 2001 and 60/374,754, filed Apr. 23, 2002. These earlier provisional applications are hereby incorporated by reference.

### FIELD OF THE INVENTION

This invention relates to a CDR-derived polypeptide from monoclonal antibody 13B8.2, pharmaceutical compositions made therefrom and methods of treating mammals with selected disorders.

### BACKGROUND

The CD4 molecule is a transmembrane glycoprotein of 58 kDa mainly expressed on the surface of mature T cells [1, 2]. CD4 is composed of four extracellular domains (D1-D4), which share homology with the immunoglobulin Vₖ region [3, 4], a transmembrane portion and a cytoplasmic tail non-covalently associated with the protein tyronsine kinase p56^{lck} [5]. The CD4 molecule acts as co-receptor for the major histocompatibility complex (MHC) class II and is a molecular partner for the T cell receptor (TcR) [6-9]. This trimolecular complex is critical for optimal activation of T cells [10-12]. Besides this physiological function, CD4 serves as a receptor for envelope glycoprotein of the human immunodeficiency virus (HIV), contributing to virus entry into cells [13].

Interactions with both MHC class 11 and gpI20 involve residues of the complementarity determining region (CDR)2-like loop in D1 of CD4[14-18]. On the opposite side of the D1 domain of CD4, the CDR3-like loop displays biological activities by acting as a target for molecules that inhibit immune response and HIV replication [19-22]. This latter role in a cascade of postbinding events has been demonstrated both by CDR3-like peptide analogs [23-27] and by anti-CDR3-like monoclonal antibodies (mAbs) such as 13B8.2 mAb [28-30]. The biological properties of 13B8.2 mAb have lead to its inclusion in phase I/II trials of HIV-infected patients. Those clinical trials using the anti-CD4 mAb 13B8.2 triggered a strong immune response to the mouse antibody, leading to a decrease in biological effects [31-33].

Clinical applications of full-length murine mAbs may be limited by their high immunogenicity, their inability to cross the blood/brain barrier, and their limited ability to penetrate cells and tissues [34]. To overcome such problems, recombinant DNA technology has been applied to redesign these foreign antibody molecules, making them more human-like, by chimerization [91] or humanization [92]. Although various systems have been described [93], the expression of complex proteins such as antibody molecules in the baculovirus/insect cell system offers marked advantages with respect to post-translatinal modifications, stability, yields and applicability. As far as we know, insect cells and baculovirus are devoid of pathogenic or toxic compounds for humans. Moreover, insect cells can be grown in protein-free medium, i.e., without mammalian contaminants, leading to easy and safe purification. These latter characteristics offer a strong advantage over the other systems for immunotherapeutic purposes. We previously described the construction of two cassette-transfer vectors for the expression and the secretion of complete chimeric IgG1 [94, 95] in insect cells infected with a double-recombinant baculovirus.

### SUMMARY OF THE INVENTION

This invention relates to an isolated CDR-derived polypeptide from monoclonal antibody 13B8.2.

This invention also relates to pharmaceutical compositions including a pharmaceutically acceptable carrier and at least the peptide described above.

This invention further relates to a method for treating a subject suffering from an autoimmune disorder, including administering to the subject a therapeutically effective amount of the pharmaceutical composition described, a method for treating a subject suffering from a transplant rejection including administering to the subject a therapeutically effective amount of the pharmaceutical composition, a method for treating a subject suffering from an HIV immunodeficiency disorder including administering to the subject a therapeutically effective amount of the pharmaceutical composition and a method for treating a subject suffering from a tumoral disorder including administering to the subject a therapeutically effective amount of the pharmaceutical composition.

### BRIEF DESCRIPTION OF THE DRAWINGS

FIG. 1 is a graph of protein content versus elution volume.

FIG. 2 is a graph of inhibition of 13B8.2 mAb/CD4 binding (%) as a function of recombinant Fab 13B8.2 (nM).

FIG. 3 is a graph of BIAcore binding analysis curves with response (RU) as a function of time (s).

FIG. 4 is an epitoped characterization of recombinant Fab and mAb 13B8.2 in accordance with the Spot method.

FIG. 5A is the inhibition percentage of IL2 secretion by pdb10fT cells sensitized with PEP 24-stimulated EBV-Lu cells and co-cultured with inhibitor antibodies.

FIG. 5B is a graph of the inhibition percentage of LTR-driven β-galactosidase gene expression induced by HIV-1ₗₐᵢ following incubation with inhibitor antibodies.

FIG. 6 is the acid amino sequences of V_{H} and V_{L} domains of the anti-CD4 mAb 13B8.2.

FIG. 7 shows a quantitative analysis of the binding of biotinylated-rhCD4 to overlapping dodecapeptides derived from the variable sequences of anti-CD4 13B8.2 mAb.

FIG. 8 is a graph showing the ELISA binding assay of His₆-sCD4 onto absorbed cyclic peptides derived from the sequence of 13B8.2.

FIG. 9 is a graph of the epitope specificity of the binding of PDPs CB1 and CB8 to sCD4 with inhibition of CD4 binding to PDPs as a function of 13B8.2 mAb concentration.

FIG. 10A is a graph of the percent inhibition of β-galactosidase activity induced by HIV-1ₗₐᵢ following incubation with cyclic PDPs.

FIG. 10B is a graph of Dose response curves for HIV-1ₗₐᵢ infected HeLa P4 cells cultured in the presence of various concentrations of 13B8.2 mAb and PDP CB1 in the form of percent inhibition of β-galactosidase activity as a function of inhibitor concentration.

### DETAILED DESCRIPTION

We developed the concept of paratope-derived peptides (PDPs) which correspond to short amino acid sequences derived from antibody variable regions and which display antigen binding and biological activities [35-38]. These small molecules are screened from a systematic exploration or antibody variable domain sequences by the Spot method [39,40]. Given the pharmaceutical interest of 13B8.2 mAb, it appeared to us attractive to design such anti-CD4 PDPs.

To this end, we chimerized the 13B8.2 mAb as a recombinant Fab fragment expressed in the baculovirus/insect cell system. The recombinant chimeric Fab 13B8.2 displays similar CD4-binding and immunosuppresive properties as the parental mouse mAb. These functional effects of chimeric Fab 13B8.2 make it a good candidate for therapeutic purposes. Therapies of particular interest include, but are not limited to, autoimmune disorder, transplant rejection, HIV immunodeficiency disorder and tumoral disorder. Preferred autoimmune disorders include, for example, psoriasis, rheumatoid arthritis and lupus arythematosus. Specifically, we identified nine PDPs from the 13B8.2 variable regions by using the Spot method. All the selected PDPs, prepared in a soluble cyclic form, were able to bind histidine-tagged recombinant CD4 (His₆-sCD4) expressed in baculovirus. MAb 13B8.2 specifically displaced the binding of His₆-sCD4 to PDPs CB1 and CB8, indicating that anti-CD4 PDPs recognize an epitope on the CD4 molecule closely related or similar to that identified for the 13B8.2 parental mAb. PDP CB1 displayed biological properties very similar to those of the parental 13B8.2 mAb, inhibiting in vitro antigen presentation and HIV-1 promoter activation. Taken together, we believe that the bioactive PDP CB1, derived from the CDR-H1 region of the anti-CD4 13B8.2 mAb, is valuable for anti-CD4 peptidomimetics.

### Materials

Reagents, Cell Lines and Vectors

Recombinant human soluble CD4 (rhCD4) was obtained from Repligen (Needham, Mass., USA). rhCD4 was biotinylated using a commercial reagent (Amersham Pharmacia Biotech, Cleveland, Ohio, USA) according to the manufacturer's instructions and stored in PBS at −20° C. until use. 13B8.2 mAb [19,31] was obtained from Immunotech-Coulter (Marseille, France). The murine hybridoma cell line that produces 13B8.2 mAb (IgG1/κ[19]) was provided by Dr. D. Olive and Dr. C. Mawas (INSERM U119, Marseille, France). The pMV7-T4 plasmid, encoding the full-length CD4-cDNA sequence [41,42], was provided by Dr. Q. J. Sattentau (Centre d'Immunologie de Marseille-Luminy, Marseille, France). The human lymphoblastoid B cell line EBV-Lu, expressing the HLA DR5,6, DRB52, DQ6,7, and A2 molecules, and the murine T cell pdb10F, expressing human CD4 and pep24 (PAGFAILKCNNKTFNY)-specific chimeric TcR, have been previously characterized [43,44] were provided by P. DeBerardinis (Consiglio Nazionale delle Ricerche, Napoly, Italy). The HeLa P4 HIV-1 LTR β-galactosidase indicator cell line [45] was provided by Dr. O. Schwartz (Institut Pasteur, Paris, France).

### EXAMPLE 1

Baculovirus Expression of Recombinant CD4

The nucleotide sequence of soluble CD4 (D1-D4) was sorted by PCR from the pMV7-T4 plasmid, then cloned into the p119L baculovirus transfert vector to allow the expression of CD4 under the P10 promoter, as described elsewhere except that no histidine tag was inserted in the construction. Transfection of Sf9 cells and further expression of CD4 in baculovirus supernatant was performed [94, 95]. An enriched-CD4 fraction was prepared following 80% ammonium sulphate precipitation of the baculovirus supernatant and subsequent dialysis in a 0.1×160 mM PBS solution.

Construction of Recombinant Baculovirus Producing the Chimeric Mouse/Human Anti-CD4 Fab 13B8.2

The general procedures concerning the cloning and sequencing of 13B8.2 mAb variable regions have been described [99]. Two plasmid cassette-transfer vectors pBHuCk and pBHuFDγ1 were constructed that contain the human Cκ gene [94] and the first domain of human Cγ₁ (Fdγ₁), allowing the insertion and expression of variable heavy (VH and kappa light (Vκ) chains of the anti-CD4 mAb 13B8.2 under the control of the polyhedrin and p10 promoter: pBHuFdγ₁ was obtained by using the same procedure as that described by Poul et al. [94] for the construction of the pBHuCγ₁ plasmid vector except that a stop codon was inserted at the end of the gene encoding for the first domain of the Cγ₁ constant region. A two-step recombination procedure [94, 95] was carried out to construct the recombinant baculovirus, named 5756, expressing both heavy and light chains of the chimeric Fab 13B8.2.

Anti-CD4 Fab 13B8.2 Production and Purification

A 400 ml-supernatant of Spodoptera frugiperda Sf9 cells (ATCC CRL 1711) infected with the recombinant baculovirus 5756 in a spinner culture 10⁶ cells/ml) was recovered 96-h post infection and precipitated with a saturated ammonium sulphate solution until 80% saturation. After centrifugation at 10,000 g for 30 min, the pellet was dissolved in 160 mM PBS, pH 7.2, and extensively dialyzed against PBS. The antibody solution, diluted v/v with 100 mM sodium acetate buffer, pH 5.0, was filtered (0.22 μm) and applied to a protein-G column )(Pierce, Rockford, Ill.) which has been equilibrated with 100 mM sodium acetate buffer, pH 5.0. Bound recombinant Fab 13B8.2 was eluted with 50 mM Glyciner-HCl buffer, pH 2.5, and immediately neutralized to pH7 with a 0.2 M Tris solution, pH 10.5. The protein content was monitored at 280 nm and purification fractions were checked for anti-CD4 activity by ELISA as described below. Samples were analyzed under reducing and non-reducing conditions on 12.5% polyacrylamide gel, according to the Laemlli procedure [100]. Proteins were subsequently transferred to a nitrocellulose membrane (Hybond ECL, Amersham Pharmacia Biotech) and detected with a peroxidase-conjugated anti-human kappa chain (Sigma) and a sheep anti-human Fdγ₁ (The Binding Site, Birmingham, UK) by using the ECL detection kit (Amersham Pharmacia Biotech).

Peptide Synthesis on Cellulose Membrane Covering the D1 Domain of CD4

The general protocol has been described previously [101]. By the Spot method, we synthesized 98 overlapping dodecapeptides frameshifted by one residue, representing the D1 domain of the CD4 molecule, on a cellulose membrane.

Assay for Recombinant Fab 13B8.2 Interaction with Cellulose-Bound Peptides Covering the D1 Domain of CD4

The saturated membrane was incubated either with a 6.25 nM solution of mouse mAb 13B8.2 or with a 50 nM solution of recombinant Fab 13B8.2 for 2 h at 37° C. Bound antibodies were detected with a 1:500 solution of either peroxidase-labeled anti-mouse IgG conjugate (Sigma, saint Louis, Mo.) or peroxidase-labeled anti-human kappa chain conjugate (Sigma), followed by ECL revelation (Amersham Pharmacia Biotech).

Binding Studies of Recombinant Fab 13B8.2 to CD4

A 1:500 dilution of the enriched-CD4 fraction in 0.1 M carbonate/bicarbonate buffer, pH9.6, was coated overnight at 4° C. onto 96-well enzyme immuno assay plates (Nunc, Paisley, United Kingdom). Four washes with 160 mM PBS, pH 7.2, containing 0.1% Tween 20 (PBS-T) were performed before and after saturating plates in 1% nonfat powdered milk in PBS-T for 1 h at 37° C. Thereafter, 100 μl of two-fold serial dilutions of the antibody solution was added to each well. Following incubation of 2 h and four washes in PBS-T, bound antibodies were detected by addition of 100 μl of a 1:1000 solution of peroxidase-conjugate anti-mouse IgG (Sigma) or peroxidase-conjugated anti-human kappa chain (Sigma), followed by subsequent addition of peroxidase substrate. Absorbance was measured at 490 nm (A490). For the inhibition of CD4 binding to mouse mAb 13B8.2 by recombinant Fab 13B8.2, a similar ELISA method was performed except that 13B8.2 mAb, at a 6.25 nM concentration giving an A490 of 1.0, was co-incubated with 2-fold serial dilutions of recombinant Fab 13B8.2. Three replicates were tested for each dilution with an initial Fab concentration of 1 μM. CD4/13B8.2 mAb residual binding was evaluated as described above.

The kinetic parameters of the binding of CD4 to 13B8.2 antibody were determined by surface plasmon resonance analysis using a BIAcore instrument (BI-Acore AB, Uppsala, Sweden). In an initial experiment, CD4 was immobilized on a CM5 sensorchip and 100 nM Mab 13B8.2 or recombinant Fab in HBS buffer (10 mM Hepes pH 7.6, 150 nM NaCl) were then injected. In a second experiment, 400 nM recombinant Fab were immobilized on a CM5 sensorchip by using the anti-human Fdγ₁ conjugate (The Binding Site). The binding kinetics were determined by injecting various concentrations of CD4 in HBS buffer. The kinetic parameters were calculated by using the BIA evaluation 3.0 software and the so-called “global” method [102].

IL-2 Secretion Assay Following Antigen Presentation

The EBV-Lu antigen-presenting cells (APC) were maintained in RPMI medium (BioWhittaker, Walkersville, Md.) supplemented with 10% FCS, 2 mM glutamine and 100 μg/ml penicillin/streptomycin (Sigma). Responder pdb10F T cells were maintained in DMEM medium (Gibco, Paisley, United Kingdom) supplemented with 10% fetal calf serum (FCS), 50 nM 2-mercaptoethanol, 10 nM HEPES, 2 mM glutamine, 100 μg/ml penicillin/streptomycin and kept under selection with 400 nM methotrexate and 900 ng/ml puromicine (Sigma). EVB-Lu cells (10⁶ cells/ml) were pulsed overnight at 37° C. with the pep24 stimulator peptide (75 μM) from HIV gp120 [97]. Cells were washed in PBS buffer without Ca²+and Mg²+(Biowhittaker) and plated at 10⁵ cells/well. Pdb10F reporter cells were washed with the same PBS buffer, diluted in DMEM medium without methotrexate and puromycin to a final concentration of 4×10⁵ cells/ml. 50 μl of cell suspension were plated onto EBV-Lu cells. Fifty microliters of inhibitor antibodies were then added to cells and antigen presentation was performed for 24 h at 37° C. Thereafter, 100 μl of supernatant was harvested and tested for IL-2 secretion using a commercial ELISA kit (Pharmingen, San Diego, Calif.). A positive control assay for IL-2 secretion was performed as described above except that the pdb10F cells were activated using a murine anti-CD3 antibody (0.6 nM, Pharmingen).

HIV-1 Promoter Activation Assay

HeLa P4 indicator cells (8×10⁴ cells/ml) were cultured in medium supplemented or not with infectious HIV-1_{Lai} in the presence or absence of antibodies for three days, harvested and lysed. β-galactosidase activity was then determined as previously described by measuring the absorbance at 410 nm [98].

Expression, Secretion and Purification of the Recombinant Fab 13B8.2 from Baculovirus-Infected Insect Cells

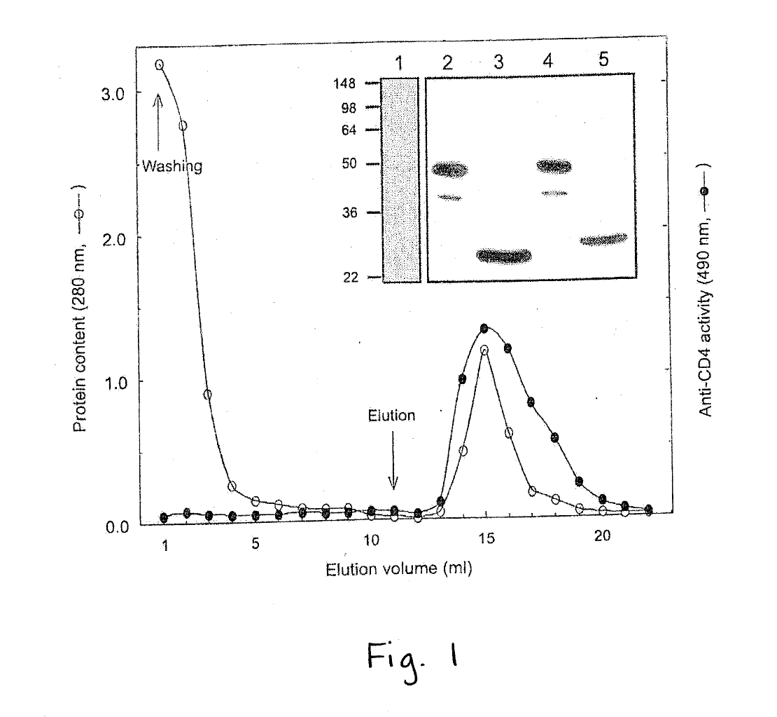

The nucleotide sequences of the VH and VL domains of 13B8.2 mAb (accession numbers AJ279001 and 279000) have been previously established according to the general procedure described by Chardè et al. [99]. Genetic analysis of these sequences [99] showed that the VH region of 13B8.2 mAb resulted from the rearrangement of VH2-DQ52-JH3 genes and that the VL region resulted from a Vκ12/13-Jκ2 gene rearrangement. In the recombinant baculovirus designated 5756, the chimeric Cκ-Vκ 13B8.2 and Fdγ₁-VH13B8.2 genes are under the control of the very late polyhedrin and p10 promoters, respectively. Recombinant Fab 13B8.2 was protein-G immunopurified from 400 ml of supernatant, obtained 96 h post infection of insect cells with 5756 baculovirus (FIG. 1). The wash fractions 1 to 10 revealed a decrease in protein content with no detectable anti-CD4 activity, whereas eluted fractions 14 to 18 showed strong anti-CD4 activity in correlation with an increase in protein content.

These fractions were further pooled for antibody analysis by Coomassie blue SDS-polyacrylamide gel electrophoresis (SDS-PAGE) and western blotting (FIG. 1, inset). Coomassie blue SDS-PAGE revealed a single band at 50 kDa, corresponding to the expected size of a correctly-processed Fab under non-reducing conditions (FIG. 1, lane 1). No other band was observed by Coomassie blue staining, demonstrating the quality of the protein-G immuno purification. The identity of the 50-kDa band was confirmed by western blotting using anti-human K chain (FIG. 1, lane 2) or anti-human Fdγ₁ chain (FIG. 1, lane 4). Individually-expressed heavy and light chains were not detected by western blotting following non-reduced SDS-PAGE. Reduction of the purified recombinant Fab 13B8.2 generated one band of approximately 25 kDa, detected by using anti-human κ chain (Fib. 1, lane 3), and one band around 28 kDa, detected by using anti-human Fdγ₁ chain (FIG. 1, lane 5). Taken together these results indicate that the anti-CD4 recombinant Fab 13B8.2 produced in the baculovirus/insect cell system is correctly assembled and secreted in the supernarant as a dimeric HL complex. The yield of purified baculovirus-expressed Fab 13B8.2 was about 5 mg/L.

Baculovirus-Expressed Chimeric Fab 13B8.2 Specifically Binds the CD4 Molecule

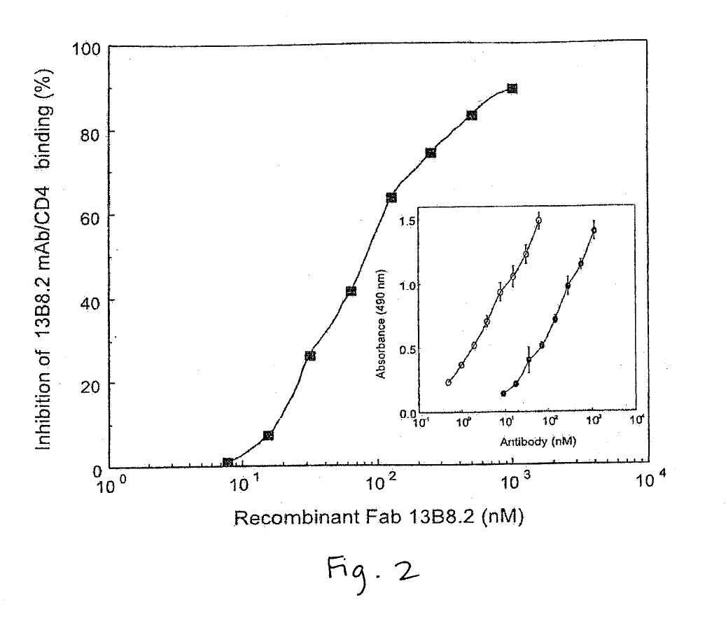

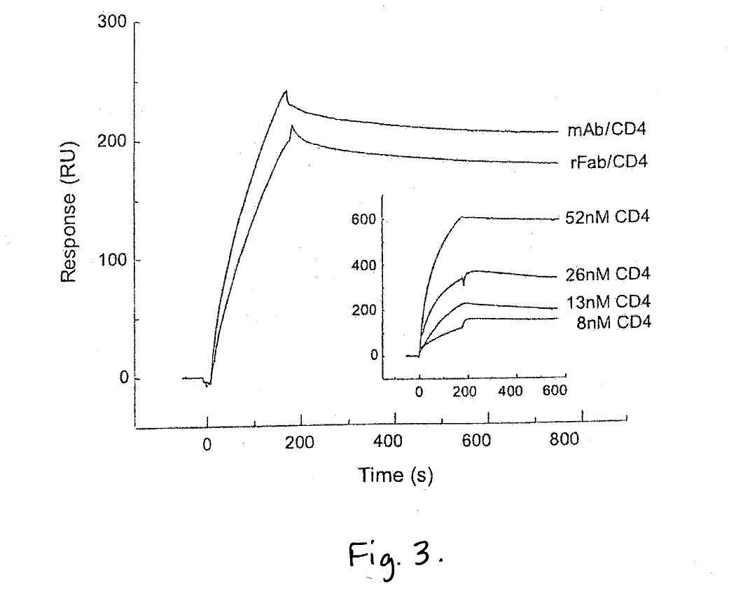

The ability of recombinant Fab 13B8.2 to bind CD4 was assessed by an ELISA method (FIG. 2) and by BIA-core analysis (FIG. 3). By ELISA, CD4 binding activity was demonstrated for a chimeric Fab concentration in the 10-1000 nM range (FIG. 2, inset), whereas no binding was obtained with the irrelevant baculovirus-expressed Fab 1C10 (data not shown). Control mAb 13B8.2 displayed CD4 binding in the 1-100 nM range (FIG. 2, inset). By using BIAcore technology (FIG. 3), CD4/Fab interaction was confirmed with a K_{D} value of 3.3 nM, whereas the affinity of the parental mAb was about 2.5 nM. This Fab interaction was CD4 dose-dependent (FIG. 3, inset). No measurable finding was obtained with the irrelevant Fab 1C10 (data not shown). Finally, the recombinant Fab 13B8.2 was able to displace the binding the parental mAb to CD4 in a dose-dependent manner (FIG. 2). A 50% inhibition of binding was obtained for a Fab concentration of 80 nM. These results indicated that the baculovirus-expressed Fab 13B8.2 finds the CD4 molecule on the same region as the parental antibody. Furthermore, the same CDR3-like region 87EDQKEEVQLLVFGLTA102 on the D1 domain of CD4 was identified as the binding region of the fragment and the intact antibody products, as shown by Spot analysis (FIG. 4), definitively demonstrating that a similar epitope is recognized by the mouse parental antibody and the recombinant Fab. No spot reactivity was observed with dodecapeptides covering the other regions on the D1 domain of CD4.

Baculovirus-Expressed Chimeric Fab 13B8.2 Displays Immunosuppressive Biological Properties

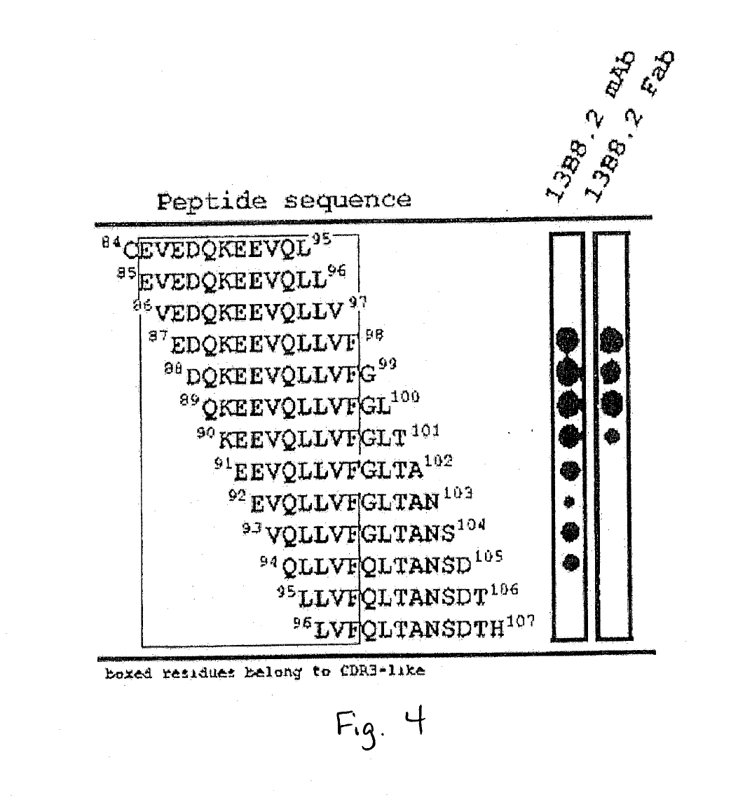

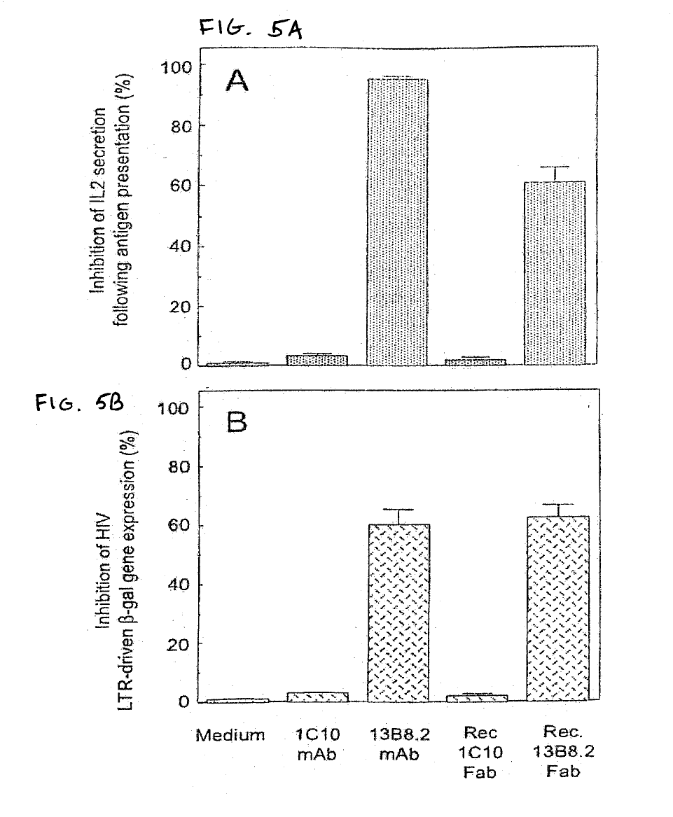

The stimulation of pdb10f responder T cells by pep24-pulsed EBV-Lu APC leads to the lymphocyte secretion of IL2[97]. This T cell activation model was found to be specific since no IL-2 secretion occurred when EBV-Lu antigen-presenting cells were pulsed with a non-stimulator pep23 antigen. The viability of pdb10f responder cell line was checked by activating cells with a murine anti-CD3 mAb which induced IL-2 secretion. As shown in FIG. 5(A), 7.8 nM of the irrelevant anti-digoxin mAb 1C10 showed no inhibitory activity of IL2 secretion in contrast to the same concentration of anti-CD4 13B8.2 mAb which blocked the IL2 production (95.7±0.7% inhibition). As compared with the irrelevant recombinant Fab 1C10 showing no inhibition, 200 nM of 13B8.2 Fab displayed 61.3±4.6% inhibitory activity of IL2 secretion. Taken together, these results indicate that, as already demonstrated for other anti-CD4 mAbs [103], the baculovirus-expressed Fab 13B8.2 is able to inhibit the antigen-presenting function, a biological property also demonstrated for the 13B8.2 parental mAb.

In order to verify the ability of recombinant Fab to inhibit HIV-1 promoter activity, as the parental 13B8.2 mAb does, we measured the β-galactosidase reporter gene expression after infection of the indicator cell line HeLa P4 cultured for three days in the presence of products. As shown in FIG. 5(B), 7.8 nM of 1C10 mAb did not display any inhibitory activity in contrast to the same concentration of aprental 13B8.2 mAb which inhibited HIV promoter activation (60.3±5.0% inhibition). Culturing infected HeLa P4 cells with irrelevant baculovirus-expressed Fab 1C10 did not affect the β-galactosidase expression, whereas significant inhibition (61.2±4.5%) was found with 200 nM of the anti-CD4 recombinant Fab 13B8.2. These results indicate that the baculovirus-expressed Fab 13B8.2 showed anti-viral property, as already demonstrated for the parental anti-CD4 mAb 13B8.2 [85, 86].

We prepared a chimeric recombinant anti-CD4 Fab expressed in baculovirus that mimics the biological properties of the parental 13B8.2 antibody. Such a functional anti-CD4 antibody provides for use of the recombinant Fab in mammals. The chimeric mouse-human Fab 13B8.2 was able to recognize a CDR3-like region in the D1 domain of CD4, comprising Glu87 and Asp88 residues previously described by site-directed mutagenesis as involved in the 13B8.2 epitope [83, 104]. IL2 secretion of activated T cells upon antigen presentation can be inhibited by the chimeric Fab, indicating that the baculovirus-expressed recombinant molecule showed immunosuppressive property classically observed for anti-CD4 antibodies [103]. As previously reported for the mouse mAb 13B8.2 [85], the recombinant anti-CD4 Fab was able to prevent HIV-1 promoter activation. This activity was probably related through the inhibition of the ERK/MEK signaling pathway as already demonstrated for the 13B8.2 mAb [105]. Taken together, these results indicate that the Fab has retained a major part of the parental 13B8.2 mAb properties and can be used without the side-effects of a mouse mAb. This is especially beneficial in pharmaceutical compositions.

The mAb 13B8.2 was shown to block gp120 binding to CD4, inhibit HIV-induced cell fusion and prevent viral production by infected cells [82]. Inhibition of viral gene transcription in HIV-cell culture and blockade of viral production from chronically-infected cells have been demonstrated following in vitro treatment with 13B8.2 antibody, these activities being obtained with HIV-1 and HIV-2 virus isolates [85]. In addition, the 13B8.2 mAb was able to elicit gp120-specific idiotypic antibody response in rabbits, thereby inhibiting syncitium formation [106]. These results led to experiments using mouse 13B8.2 antibody for HIB-1-infected patients [87-89]. Although these experiments led to clinical benefit for the patients, i.e., disappearance of circulating p24 together [87, 89] with negativation of the reverse transcriptase assay [87] or generation of serum antibodies to gp120 and HIV-1 neutralizing antibodies [89], adverse effects such as an anti-allotype or -isotype response to the foreign antibody [87, 89] or CD4⁺ clearance by the Fc portion of the antibody [87, 89] have also been documented. In sharp contrast, the chimeric Fab of this invention, showing antiviral activity like 13B8.2 mAb [83, 85, 86], is a valuable tool to overcome such problems.

Some anti-CD4 antibodies have already been used in the treatment of various autoimmune diseases or allograft rejection [107-110]. Early clinical trials using murine anti-CD4 mAbs [111] have often been discouraging, with mild tolerance of the antibody preparation and a systematic human anti-mouse response. This latter side-effect leads to the clearing of the infused mAb and induction of anaphylactic responses [112]. One way to overcome these undesirable effects has been the generation of human mAb from human immunoglobulin transgenic mice [113]. Several strategies using DNA technology have also been described, such as primatization in which anti-CD4 variable regions (V-regions) from antibody generated in macaques are fused to human constant regions [114], humanization by grafting of murine anti-CD4 CDRs inside human antibodies [92], chimerization in which murine anti-oCD4 V-regions are co-expressed with human constant domains [91]. These re-designed recombinant anti-CD4 molecules demonstrated efficient immunosuppressive activity for treatment of autoimmune diseases or transplant rejection and were shown to be devoid of the adverse side-effects of mouse antibodies. In a similar manner, the engineered chimeric anti-CD4 Fab 13B8.2, which inhibits antigen presentation, is a potent immunosuppressive agent that can be used against autoimmune diseases or for allograft rejection. Furthermore, the Fab format could overcome prolonged CD4⁺ cell depletion, a negative effect often described following in vivo treatment with whole anti-CD4 antibody [115, 116]. This effect has been attributed to synergy between complement binding capacity and Fe receptor binding on phagocytic cells [114]. Since a Fab molecule lacks the second and third domains of the heavy chain constant region which bear complement and Fc binding abilities, the chimeric Fab fragment might be devoid of cell-depleting potential, as is also the case for F(ab)′₂ fragments which block immune response to coadministered antigens and prevent the development of spontaneous autoimmune conditions [117]. Our anti-CD4 recombinant Fab probably acts as a pure receptor antagonist, either blocking CD4 receptor function or modulating the CD4 molecule on the T lymphocytes.

The baculovirus/insect cell expression system is an interesting way to produce antibodies for therapeutic purposes [118] because of its capability for a high production level [119] and the absence of known intrinsic or secreted molecules toxic for man. This is in contrast with E. coli which can release endotoxins or plants which contain toxic or allergenic compounds [119]. In addition, the ability of insect cells to grow without serum avoids the presence of mammalian contaminants [119], leading to safe and secure antibody preparation. Furthermore, the development of baculovirus surface display [120-124] coupled with a non-lytic insect cell expression system [125] might increase the applicability of this alternative eukariotic source for the generation of human antibodies.

The baculovirus/insect cell expression system allowed the production and purification of active recombinant Fab directed against the CDR3-like region of the D1 domain of CD4. This chimeric molecule retained the CD4 binding activity and the immunosuppressive properties of the parental mAb 13B8.2, thereby demonstrating its usefulness for pharmaceutical compositions.

### EXAMPLE 2

Cloning and Sequencing of 13B8.2 mAB V_{H} and V_{L} Genes

The general procedures concerning the cloning and sequencing of 13B8.2 mAb variable regions have been described [46].

Peptide Synthesis on Cellulose Membranes

202 overlapping dodecapeptides frameshifted by one residue representing the V_{H} and V_{L} sequences of 13B8.2 mAb on a cellulose membrane were synthesized according to previously described protocols [37].

Assay for sCD4 Interaction with Cellulose-Bound Peptides

The saturated membrane was incubated with a 20 nM solution of biotinylated-rhCD4 for 2 h at 37° C. Bound biotinylated-rhCD4 was detected by incubating the membrane for 1 h at 37° with a 1:3000 solution of alkaline phosphatase-conjugated streptavidin (Sigma, St. Louis, Mo., USA) and subsequent addition of 5-bromo-4-chloro-3-indolyl phosphate substrate. Inhibition of biotinylated-rhCD4 binding was performed as described above, except that biotinylated-rhCD4 (20 nM) was pre-incubated for 18 h at 4° C. with 13B8.2 mAb (6.25 μM). In all cases, the reactivity of the spots was evaluated by scanning the membrane and measuring the intensities of the spots with the NIH image 1.61 software [39].

Synthesis of Soluble Peptides

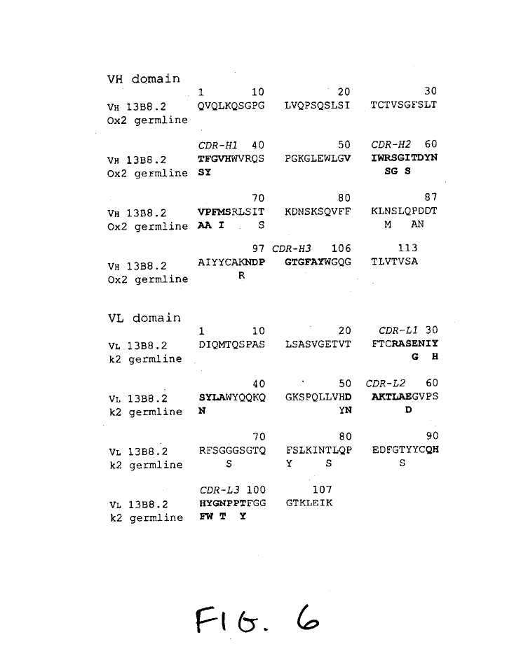

The nine selected PDPs, named CB1 to CB9 (FIG. 6, right panel for sequences), a scrambled form of PDP CM9 (ScCM9: GSDQWNKMQYYP) [35], a scrambled form of the CD4-derived CDR3-like peptide (ScCDR3-like: KEEICEVEDQTY), and an unrelated peptide (Dig97c: FGDYYCLQYASS, derived from the CDR-L3 region of the anti-digoxin 1C10 mAb), with Lys-Cys residues added to both carboxyl- and amino-termini of all peptides, were synthesized by Fmoc solid-phase synthesis on an AMS422 robot (Abimed, Langelfeld, Germany), cyclized and purified as described previously [35]. Lys and Cys residues were added, respectively, to improve the solubility and permit cyclization of the peptides. The peptides showed homogeneity in high performance liquid chromatography at the expected monomeric molecular weight. Thereafter, the peptides were resuspended in deionized water, except for the scCDR3-like peptide which was suspended in a 10% acetonitrile solution and the CB8 PDP in a 20% acetonitrile solution.

Baculovirus Expression of Recombinant His₆-sCD4

The nucleotide sequence of soluble CD4 (D1-D4) was sorted by polymerase chain reaction from the pMV7-T4 plasmid by using the sense primer SCD4FB (5′-GAAGATCTATGAACCGGGGAGTCC), which matches codons 1 to 6, and the anti-sense primer SCD4RTB (5′-GAAGATCTTCAATGGTGATGGTGGTGGTGACCTAATGCGGCCATTGGCTGCACCGGG), which contains the reverse complement of codons 367 to 372 of CD4 and encodes the His₆-tag and a BglII restriction site. Following sub-cloning into the pGEM-T vector (Promega, Madisoin, Wis., USA), the sCD4 sequence was verified by using the dideoxy termination method with the T7 sequencing kit (Pharmacia, Uppsala, Sweden). The BglII-linearized His₆-sCD4 fragment was cloned into the p119L baculovirus transfer vector to allow the expression of His₆-sCD4 under the P10 promoter. After transfection of Spodoptera frugiperda Sf9 cells (ATCC CRL 1711), recombinant baculoviruses were further purified by using a plaque assay and propagated in Sf9 cells [47,48]. Supernatant of Sf9 cells infected with the His₆-sCD4 recombinant baculovirus in a spinner culture (10⁶ cells/ml) were harvested 6 days post-infection and clarified by centrifugation at 1000×g for 5 min.

Purification and Characterization of His₆-sCD4

Purification of the His₆-sCD4 product was carried out by using Ni-NTA agarose beads (Qiagen, Chatswroth, Calif., USA) according to the manufacturer's procedure with minor modifications. Briefly, the clarified baculovirus supernatant was dialyzed against washing buffer (50 mM NaH₂PO₄, 500 mM NaCl, 5 mM imidazole) for 24 h at 4° C. Ni-NTA agarose beads were then added to a final concentration of 5%, and the binding of His₆-sCD4 was performed for 18 h at 4° C. Beads were washed with 8 volumes of washing buffer and His₆-sCD4 was eluted as 1 ml fractions with 3 volumes of elution buffer (50 mM NaH₂PO₄, 500 mM NaCl, 300 mM imidazole). The Ni-NTA agarose beads were regenerated with 1 M imidazole. All purification fractions were checked for the presence of His₆-sCD4 by enzyme-linked immunosorbent assay (ELISA) and Western blot using the anti-CD4 13B8.2 mAb as detection reagent.

Binding Studies of PDPs to CD4

Three replicates corresponding to 10-fold serial dilutions of the nine cyclic PDPs (CB1-CB9) were coated overnight at 4° C. onto 96-well ELISA plates (Nunc. Paisley, UK) with an initial peptide concentration of 50 μM. Four washes in 160 mM PBS, pH 7.2, containing 0.1% Tween 20 (PBS-T) were performed before and after saturating the wells with 1% non-fat dry milk in PBT-T for 1 h at 37° C. Thereafter, 100 μl of 20 nM His₆-sCD4 was added to each well. Following a 2 h incubation and four washes in PBS-T, bound His₆-sCD4 was detected by addition of 100 μl of a 1:2000 solution of peroxidase-conjugated anti-His₆ mAb (Sigma) and subsequent addition of peroxidase substrate. Absorbance was measured at 490 nm.

Binding Specificity of PDPs CB1 and CB8 to CD4

Inhibition of His₆-sCD4 binding to PDPs was performed by an ELISA method with PDPs CB1 and CB8 coated at 12.5 and 2.5 μM, respectively, as capture reagent. A 20 nM solution of His₆-sCD4 showing an absorbance at 490 nm of 1.0 was co-incubated with two-fold serial dilutions of 13B8.2 mAb. Three replicates were tested for each dilution with an initial mAb concentration of 3 μM. His₆-sCD4 binding was evaluated as described above.

Interleukin-2 (IL-2) Secretion Assay Following Antigen Presentation

EBV-Lu antigen-presenting cells (10⁶ cells/ml), overnight pulsed with the pep24 stimulator peptide (75 μM) from HIV gp120 [44], were co-cultured with pdb10f responder cells (4×10⁵ cells/ml) in the presence or absence of inhibitor PDPs or mAbs for 24 h. Thereafter, 100 μl of supernatant was harvested and tested for IL-2 secretion using an ELISA commercial kit (Pharmingen, San Diego, Calif., USA). A positive control for IL-2 secretion was performed as described above except that activation of pdb10F cells was done using a murine anti-CD3 antibody at a concentration of 0.6 nM (Pharmingen) [49].

HIV-1 Promoter Activation Assay

HeLa P4 indicator cells (8×10⁴ cells/ml) were cultured in medium supplemented or not with 1000 TCID₅₀ of infectious HIV-1_{Lai} in the presence or absence of peptides or mAb for 3 days, harvested and lysed. The β-galactosidase activities were then determined as previously described [45] by measuring the absorbance at 410 nm.

Characterization of Nine Peptides from 13B8.2 mAb Demonstrating CD4 Binding Ability

The nucleotide sequences of VH and VL domains of 13B8.2 mAb were established according to the general procedure described by Chardès et al. [46] and made available from the EMBL database under the accession numbers AJ279001 and AJ27900, respectively. The complete amino acid sequences of both chains are given in FIG. 6 with somatic mutations indicated. Genetic analysis of these sequences showed that the V_{H} region of 13B8.2 mAb resulted from the rearrangement of V_{H}2-DQ52-J_{H}3 genes and that the V_{L} region resulted from a V_{κ}12/13-J_{κ}2 gene rearrangement. More precisely, computer-assisted comparison of these sequences showed that the V_{H} and V_{L} genes of 13B8.2 displayed significant homologies with Ox2 [50] and k2[51] germline genes, respectively (FIG. 6). It is worth noting that no significant homology was found between 13B8.2 mAb variable sequences and other anti-CD4 variable domains.

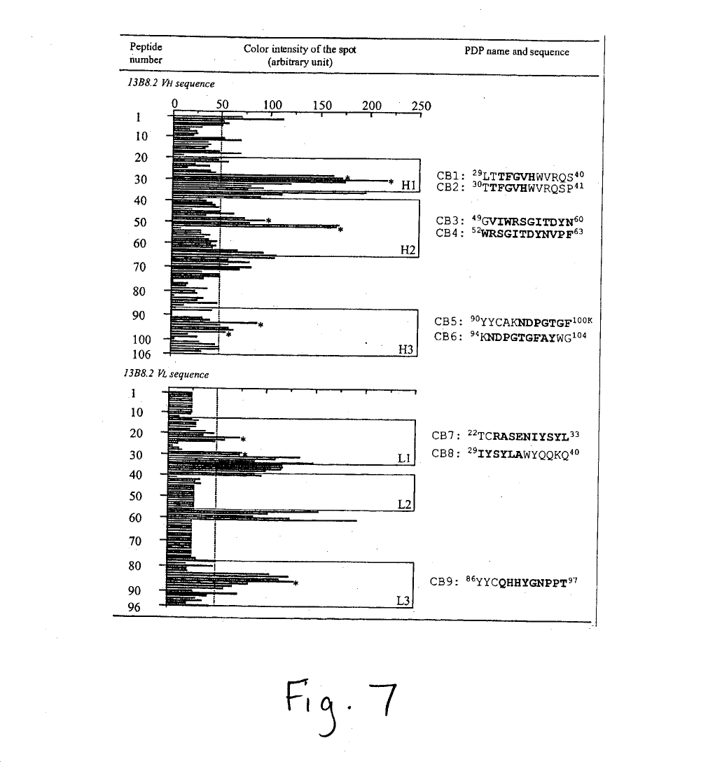

202 overlapping dodecapeptides frameshifted by one residue, corresponding to the deduced amino acid sequence of V_{H} and V_{L} from 13B8.2 mAb, were synthesized on a cellulose membrane by using the Spot method. The anti-CD4 immunoreactivity of these peptides was assessed by incubating the membrane with biotinylated-rhCD4. The results are quantitatively expressed in FIG. 7 (left panel) in which the reactivity of peptides that comprise at least one residue from the CDRs are boxed. Anti-CD4 reactivity was observed for peptides including amino acids from five of the six CDRs of 13B8.2 mAb (peptides 20, 22, 28-35; 46, 48-52, and 93-97 for CDR-H1, CDR-H2 and CDR-H3, respectively, and 22, 23, 29-34; 83-89 and 91 for CDR-L1 and CDR-L3, respectively). Anti-CD4 activity was also obtained for peptides containing residues from the framework, mainly flanking the CDRs (peptides 18, 36-38 and 66-71 for V_{H}, and 35-38 and 57-61 for V_{L}) but the majority of peptides comprising only framework residues did not display any significant binding activity. This reactivity was drastically decreased when biotinylated-rhCD4 was pre-incubated with an excess of 13B8.2 parental mAb. Nine peptides (29, 30, 48, 52, 90 and 94 for V_{H} and 22, 29 and 86 for V_{L}), named CB1 to CB9 (FIG. 2, right panel), were selected for further study in a soluble form. These peptides, except for 22 and 29 from CDR-L1, showed the highest anti-CD4 activity and comprised at least 50% of residues belonging to CDRs. Since the most reactive peptides derived from the CDR-L1 showed less than 50% of residues from the CDR (peptides 31 and 34), We selected two adjacent reactive peptides, namely, 22 and 29. Except for PDP CB4, that exclusively comprised amino acids from the CDR-H2, all selected PDPs comprised amino acids from both CDR and framework sequences.

Soluble Cyclized Selected PDPs Demonstrate CD4 Binding Activity

Peptides selected according to the Spot results were synthesized and N- to C-terminus cyclized through cysteine oxidation. Cyclization has already been demonstrated as a useful tool to improve antigen binding [36]. The ability of recombinant His₆-sCD4 expressed in baculovirus to specifically bind cyclic PDPs was assessed by ELISA (FIG. 8). The selected PDPs, except CB3 from the CDR-H2, showed a dose-dependent CD4 binding activity. PDPs CB1 and CB2 derived from the 13B8.2 CDR-H1 region, and PDP CB8 derived from the 13B8.2 CDR-L1 region, displayed high binding activity in a 0.5-50 μM concentration range. The non-reactivity of irrelevant ScCDR3-like and ScCm9 peptides indicated that the addition of lysine and cysteine residues for solubilization and cyclization of peptides had no effect on CD4 binding. Since PDPs CB1 and CB2 only differ by one amino acid residue, we focused our attention on PDP CB1, derived from the CDR-H1, and PDP CB8, derived from the CDR-L1 of 13B8.2 mAb for further specificity studies.

PDPs CB1 and CB8 Specifically Bind to CD4 on the same Region as that of Parental 13B8.2 mAb

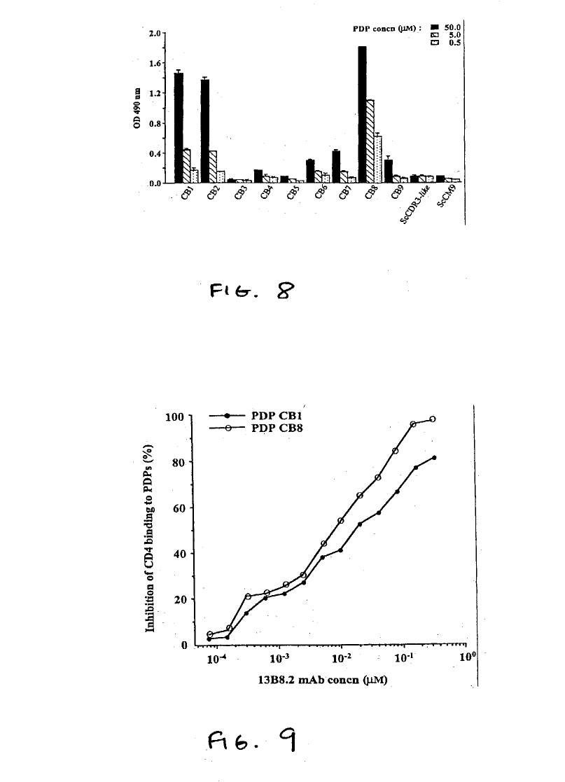

The ability of the parental mAb 13B8.2 to displace the binding of PDPs CB 1 and CB8 to His₆-sCD4 was studied by using an ELISA inhibition assay. The absorbance of residual His₆-sCD4 binding to PDPs was measured at 490 nm and expressed as percent inhibition of the binding (FIG. 9). We found that 13B8.2 mAb was able to displace the binding of His₆-sCD4 (20 nM), in a dose-dependent manner, to both coated PDPs CB1 (12.5 μM) and CB8 (2.5 μM) with similar efficiencies. A 50% inhibition of binding PDPs CB1 and CB8 to CD4 was obtained for 13B8.2 mAb concentrations of 20 and 8 nM, respectively. No inhibition was found when using the IgG1 isotype-unrelated anti-digoxin mAb 1C10, demonstrating the specificity of the competition studies.

Similar results were obtained in a symmetric experiment in which the binding of His₆-sCD4 (20 nM) to 13B8.2 mAb (0.3 nM) was inhibited by various concentrations of PDPs CB1 and CB8. A 50% inhibition of binding of His₆-sCD4 to 13B8.2 mAb wase obtained for concentrations of PDPs CB1 and CB8 up to 75 and 125 μM, respectively. Taken together, these data are consistent with the hypothesis that 13B8.2 mAb, CB1, and CB8 peptides recognized the same antigenic region on the CD4 molecule.

PDP CB1 is able to Inhibit IL-2 Secretion Following Antigen Presentation

The stimulation of pdb10f responder T cells by pep24-pulsed EBV-Lu APC leads to the lymphocyte secretion of IL2[43,44]. This T cell activation model is specific since no IL-2 secretion occurs when EBV-Lu antigen-presenting cells are pulsed with a non-stimulator pep23 antigen. We checked the viability of pdb10f responder cell line by activating cells with a murine anti-CD3 mAb which induced IL-2 secretion (data now shown). As shown in Table 1, the irrelevant anti-digoxin mAb 1C10 showed no inhibitory activity of IL2 secretion in contrast to the anti-CD4 13B8.2 mAb which blocked the IL2 production (99.6±0.2% inhibition). As compared to the irrelevant ScCM9 peptide showing no inhibition, PDP CB1 displayed inhibitory activity of IL2 secretion in a 125-250 μM concentration range. The biological activity of PDP CB2 was found to be very moderate since no activity was found at a concentration lower than 250 μM.

The lack of activity for PDP CB8 was found to be consecutive to cell death, probably due to the presence of 20% acetonitrile in the buffer used to solubilize the peptide. The other selected PDPs demonstrated no or extremely low blocking of IL2 secretion. Taken together, these results indicate that, as already demonstrated for other anti-CD4 mAbs [52,53], the CDR-H1-derived PDP CB1 is able to inhibit the antigen-presenting function, a biological property also demonstrated for the 13B8.2 parental mAb.

PDP CB1 Displays a Strong Capacity to Inhibit HIV-1_{Lai} LTR-Driven β-Galactosidase Reporter Gene Expression

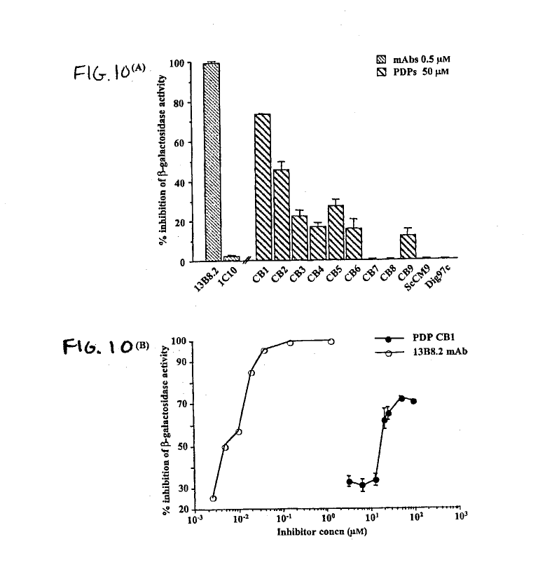

The parental mAb 13B8.2 has been previously demonstrated to be an inhibitor of viral particle production by cells infected with HIV-1_{Lai}, HIV-1_{Eli}, HIV-1_{Sf2}, HIV-1_{Ger} and HIV-2_{Rod} strains [13,54]. In addition, viremia negativation has been observed for HIV-infected patients treated with 13B8.2 antibody, demonstrating its efficiency towards primary clinical isolates [31,32]. In order to assess the ability of selected 13B8.2 PDPs to inhibit HIV-1 promoter activity, we measured the β-galactosidase reporter gene expression after infection of the indicator cell line HeLa P4 cultured for 3 days in the presence of peptides. As shown in FIG. 10A, the 1C10 mAb did not display any inhibitory activity in contrast to the parental 13B8.2 mAb which inhibited HIV promoter activation. Culturing infected HeLa P4 cells with irrelevant Dig97c or ScCm9 peptides did not affect β-galactosidase expression, whereas a significant inhibition was found when the cells were cultured with a 50 μM solution of 13B8.2 PDPs CB1, CB2 and CB5.

The lack of activity for PDP CB8 was found, in this assay, also to be consecutive to cell death caused by the presence of 20% acetonitrile in the buffer used to solubilize the peptide since no inhibition was observed with the PDP CB1 diluted in the same 20% acetonitrile buffer. In FIG. 10B, we demonstrated that the inhibitory effect of CB1 is dose-dependent, with an IC50 for CB1 of about 15 μM, whereas the IC50 of the parental mAb was found to be 5 nM. These results indicate that the CDR-H1-derived dodecapeptide CB1 has anti-viral activity, as already demonstrated for the parental anti-CD4 13B8.2 mAb.

The CD4 molecule plays a key role both in the MHC class II-restricted immune response and the human immunodeficiency virus infection process by acting as a receptor either for the TcR-antigen engagement complex or for the envelope glycoprotein gp120 of HIV [7,9,55,56]. In these two cases, CD4 has been demonstrated to induce signal transduction leading to T cell activation [12,19,21,22]. Both of these mechanisms can be inhibited by treatment with anti-CD4 mAbs including murine 13B8.2 mAb [28-30]. Such inhibitory properties have lead to the use of 13B8.2 mAb in HIV-infected patients [31-33]. To avoid problems encountered when using mAbs in therapeutic approaches, such as immunogenicity and low tissue diffusion, we designed and synthesized PDPs from the 13B8.2 anti-CD4 mAb.

We demonstrated that the CDR-H1-derived PDP CB1 displays significant and specific biological properties mimicking those of the parental anti-CD4 13B8.2 mAb. First, in an in vitro model of MHC class II-restricted immune response, we demonstrated that anti-CD4 PDP CB1, as well as parental mAb 13B8.2, inhibits IL2 secretion by activated T cells following antigen presentation. This inhibitory effect, classically observed for anti-CD4 mAbs [52,53], was dose-dependent. Since anti-CD4 mAbs have been described to prevent T cells from IL2-induced proliferation and B cell adhesion through inhibition of Ca²⁺ and P21^{ras} signaling pathways [57,58], it remains to be assessed whether our anti-CD4 PDPs could interfere with such mechanisms. The effect of CB1 in an in vivo model of immune disorder remains to be investigated, as it was done for anti-CD4 CDR3-like-derived analogs in a murine experimental allergic encephalomyelitis model [59]. Second, we found that the PDP CB1 inhibited HIV-1 promoter activation, as 13B8.2 mAb does [29]. The mechanism by which 13B8.2 mAb exerts this anti-HIV property has been related to inhibition of signal transduction, involving the extracellular regulated kinase/mitogen-activated protein kinase kinase signaling pathway [13]. We also believe that CB1 acts by disrupting the same signal transduction machinery, thereby preventing HIV pro-virus transcription.

We found that peptides CB1 and CB8 displayed specific anti-CD4 binding activity. A relatively high concentration of peptides is preferred to achieve efficient biological activity with regard to the efficient antibody dose. To explain this difference, we measured the binding of CD4 to PDP CB1 and observed a 50% decrease in binding when following pre-incubation of the peptide for 25 min at 37° in a buffer containing 10% fetal calf serum (data not shown). This finding strongly suggests that the high concentrations of CB1 which achieve a biological effect reflects degradation of the peptide in the culture medium. We believe that use of D-amino acids for peptide synthesis would improve their metabolic stability [60].

We believe that a longer sequence of PDP CB8 including hydrophilic flanking residues will improve the solubility of the peptide and avoid the use of organic solvent. This is of great interest because of a high sequence/position homology between this CDR-L1-derived PDP CB8 and a previously characterized CDR-L1-derived PDP, the CM9 peptide, derived from the anti-CD4 ST40 mAb that inhibits HIV transcription [35]. Comparison of alanine-scanning analysis of these two peptides indicated that similar residues located at the same positions, i.e. Tyr³², Trp³⁵, Tyr³⁶, Lys³⁹, contribute to CD4 binding [36]. This may help us understand the structure-function relationship in these series of anti-CD4 mAb-derived bioactive molecules.

The systematic exploration of the 13B8.2 mAb paratope has led us to the characterization of PDPs CB1 and CB8, displaying high anti-CD4 reactivity and including residues from the CDRs and from the framework flanking these CDRs. The role of residues outside the CDRs (i.e. Trp³⁶ and Arg³⁸ in the CB1 sequence, and Tyr³⁶ in the CB8 sequence) has already been described as being important in structuring the active CDR loops in the full mAb paratopes [61,62]. Moreover, Park et al. [63] demonstrated the relevant importance of adding aromatic residues to improve the efficiency of their CDR-H3-derived anti-HER2/neu peptide mimetics. The addition of aromatic residues has been found to be a valuable strategy for enhancing stability, folding, and avidity of peptidomimetics [25,64-66]. This could explain the high reactivity obtained for PDP CB1, in which the natural Trp³⁶ residue may have such function. We demonstrated differences, both in anti-CD4 binding activity and in biological properties, between PDPs CB1 and CB2, that are frameshifted by only one residue. Two key points may explain these differences. First, PDP CB2 contains the Pro⁴¹ residue which may constrain the peptide in an unfavorable conformation. Second, in PDP CB2 the Leu²⁹ residue is absent. The Leu²⁹ residue is part of the Vernier zone [67], already described to stabilize Ag/Ab interactions. These two factors may contribute to twisting PDP CB2 into a less favorable conformation for CD4 binding, thereby explaining the decreased bio-logical properties of CB2. Taken together, these data confirm the capacity of the Spot method in defining antigen-specific peptides derived from a mAb paratope that present paratope-derived residues in an environment compatible for antigen binding.

The CDR-3-like loop of domain 1 of CD4, and more precisely the negatively charged residues Glu⁹¹ and Glu⁹², has been shown to play a role in activating T cell signal transduction [20,21]; Glu⁹¹ and Glu⁹² are also involved in the 13B8.2 paratope [16]. Inhibition studies demonstrated that PDP CB1 and 13B8.2 mAb specifically compete for CD4 binging on the same region of the molecule. Alanine scanning of the CB1 sequence showed that the main contributor residues are positively charged (i.e., His³⁵ and Arg³⁸, unpublished data), thereby possibly interacting with its negatively charged epitope on the CD4 molecule. The CDR3-like loop from the D1 domain of CD4 has been reported to be involved in on of the two potential CD4 dimerization sites [20,21,68,69]. The dimerization/oligomerization processes have been shown to be necessary for optimal activation of CD4⁺ T lymphocytes [16,26,70-72]. It is, therefore, possible that the inhibitory effects of PDP CB1 on T cell stimulation and HIV-1 promoter activation can result from a CD4 dimerization/obligomerization disruption, thereby uncoupling the CD4 molecule from the signal transduction machinery. Even if this molecular mechanism remains to be established, our approach has led to the characterization of the anti-CD4 PDP CB1. This kind of small bioactive molecule is useful for more potent and stable molecules of pharmaceutical interest.

### REFERENCES

[1] Littman, D. R. (1987) Annu. Rev. Immunol. 5, 561-584.

[2] Muddon, P. J., Molineaux, S. M., Maddon, D. E., Zimmerman, K. A., Godfrey, M., Alt, F. W., Chess, L. and Axel, R. (1987) Proc. Natl. Acad. Sci. USA 84, 9155-9159.

[3] Maddon, P. J., Dalgleish, A. G., McDougal, J. S., Clapham, P. R., Weiss, R. A. and Axel, R. (1986) Cell 47, 333-348.

[4] Wang, J. H., Yan, Y. W., Garrett, T. P., Liu, J. H., Rodgers, D. W., Garlick, R. L., Tarr, G. E., Husain, Y., Reinherz, E. L., and Harrison, S. C. (1990) Nature 348, 411-418.

[5] Rudd, C. E., Trevillynn, J. M., Dasgupta, J. D., Wong, L. L. and Schlossman, S. F. (1988) Proc. Natl. Acad. Sci. USA 85, 5190-5194.

[6] Konig, R., Huang, L. Y. and Germain, R. N. (1992) Nature 356, 796-798.

[7] Brogdon, J., Eckels, D. D., Davies, C., White, S. And Doyle, C. (1998) J. Immunol. 161, 5472-5480.

[8] Dianzani, U., Shaw, A., al-Ramadi, B. K., Kubo, R. T. and Janoway Jr., C. A. (1992) J. Immunol. 148, 678-688.

[9] Vignali, D. A. and Vignali, K. M. (1999) J. Immunol. 162, 1431-1439.

[10] Sakihama, T., Smolyar, A. And Reinherz, E. L. (1995) Immunol. Today 16, 581-587.

[11] Hampl, J., Chien, Y. H. and Davis, M. M. (1997) Immunity 7, 379-385.

[12] Grakobi, A., Bromley, S. K., Sumen, C., Davis, M. M., Shaw, A. S., Allen, P. M. and Dustin, M. L. (1999) Science 285, 221-227.

[13] Briant, L. and Devaux, C. (2000) Adv. Pharmacol. 48, 373-407.

[14] Clayton, L. K., Sieh, M., Pious, D. A. and Reinherz, E. L. (1989) Nature 339, 548-551.

[15] Moebius, U., Pallai, P., Harrison, S. C. and Reinherz, E. L. (1993) Proc. Natl. Acad. Sci. USA 90, 8259-8263.

[16] Houlgatte, R., Scarmato, P., el Marbomy, S., Martin, M., Ostankovitch, M., Lafosse, S., Vervisch, A., Auffray, C., and Piatier-Tonneau, D. (1994) J. Immunol. 152, 4475-4488.

[17] Sakihama, T., Smolyar, A. and Reinherz, E. L. (1995) Proc. Natl. Acad. Sci. USA 92, 6444-6448.

[18] Huang, B., Yachou, A., Fleury, S., Hendrickson, W. a. and Sekaly, R. P. (1997) J. Immunol. 158, 216-225.

[19] Corbeau, P., Benkirane, M., Weil, R., David, C., Emiliani, S., Olive, D., Mawas, C., Serre, A. and Devaux, C. (1993) J. Immunol. 150, 290-301.

[20] Friedman, T. M., Reddy, A. P., Wassell, R., Jameson, B. A. and Korngold, R. (1996) J. Biol. Chem. 271, 22635-22640.

[21] Briant, L., Signoret, N., Gaubin, M., Robert-Hebmann, V., Zhang, X., Murali, R., Greene, M. L., Piatier-Tonneau, D. and Devaux, C. (1997) J. Biol. Chem. 272, 19441-19450.

[22] Simon, J. H., Stumbles, T., Signorel, N., Somoza, C., Puklavec, M., Sattentau, Q. J., Barclay, A. N. and James, W. (1997) J. Virol 71, 1476-1484.

[23] Ohki, K., Kumagai, K., Mitsuda, S., Takano, T., Kimura, T. and Ikuta, K. (1993) Vaccine 11, 682-686.

[24] Kumagai, K., Tokonaga, K., Tsutsarni, M. and Ikata, K. (1993) FEBS Lett. 330, 117-121.

[25] Zhang, X., Piatier-Tonneau, D., Auffray, C., Murali, R., Mahapatra, A., Zhang P., Maier, C. C., Saragovi, H. and Greene, M. I. (1996) Nat. Biotcchnol. 14, 472-475.

[26] Koch, U., Choksi, S., Marcucci, L. and Komgold, R. (1998) J. Immunol. 161, 421-429.

[27] Roland, J., Berezov, A., Green, M. I., Murali, R., Piatier-Tonneau, D., Devaux, C. and Briant, L. (1999) DNA Cell Biol. 18, 819-828.

[28] Truneh, A., Buck, D., Cassatt, D. R., Jaszczak, R., Kassis, S., Ryu, S. E., Hcaley, D., Sweet, R. and Sattentau, Q. (1991) J. Biol. Chem. 266, 5942-5948.

[29] Benkirane, M., Corbeau, P., Housset, V. and Devaux, C. (1993) EMBO J. 12, 4909-4921.

[30] Benkirane, M., Schmid-Antomarchi, H., Littman, R. D., Him, M., Rossi, B. and Devaux, C. (1995) J. Virol. 69, 6904-6910.

[31] Dhiver, C., Olive, D., Rousseau, S., Tarnalet, C., Lopez, M., Galindo, J. R., Mourens, M., Him, M., Gastaut, J. A. and Mawas, C. (1989) AIDS 3, 835-842.

[32] Deckert, P. M., Ballmaier, M., Lang S., Deicher, H. and Schedel, T. (1996) J. Immunol, 156, 826-833.

[33] Schedel, I., Sutor, G. C., Hunsmann, G. and Jurkiewicz, E. (1999) Vaccine 17, 1837-1845.

[34] Cho, M. J. and Juliano, R. (1996) Trends Biotechnol. 14, 153-158.

[35] Monnel, C., Laune, D., Laroche-Trameau, J., Biard-Piechaczyk, M., Briant, L., Bés, C., Pugniere, M., Mani, J. C., Pau, B., Cerulti, M., Devauchelle, G., Devaux, C., Grainer, C. and Chardes, T. (1999) J. Biol. Chem. 274, 2789-3796.

[36] Pugniere, M., Charde, T., Bes, C. Monnet, C., Laune, D., Granier, C. and Mani, J. C. (2000) Int. J. Bio-chromatogr. 5, 187-197.

[37] Laune, D., Molina, F., Ferrieres, G., Mani, J. C., Cohen, P., Simon. D., Bemardi, T., Piechaczyk, M., Pau, B. and Granier, C. (1997) J. Biol. Chem. 272, 30937-30944.

[38] Laune, D., Pau, B. and Granier, C. (1998) Clin. Chem. Lab. Med. 36, 367-371.

[39] Molina, P., Laune, D., Gougat, C., Pau, B. and Granier, (1996) Pept. Res. 9, 151-155.

[40] Franck, R. (1992) Tetrahedron 98, 9217-9232.

[41] Maddon, P. J., Littman, D. R., Godfrey, M., Muddon, D. E., Chess, L. and Axel, R. (1985) Cell 42, 93-104.

[42] Kirschmeier, P. T., Ilousey, G. M., Johnson, M. D., Perkins, A. S. and Weinstein, I. B. (1988) DNA 7, 219-225.

[43] Blank, U., Boitel, B., Mege, D., Ermonval, M. and Acuto, 0. (1993) Eur. J. Immunol. 23, 3057-3065.

[44] Manca, F., De Berardinis, P., Fenoglio, D., Ombra, M. N., Li Pira, G., Saverino, D., Autiero, M., Lozzi, L., Bracci, L. and Guardiola, J. (1996) Eur. J. Immunol. 26, 2461-2469.

[45] Briant, L., Robert-Hebmann, V., Acquaviva, C., Pelchen-Matthews, A., Marsh, M. and Devaux, C. (1998) J. Virol. 72, 6207-6214.

[46] Chardes, T., Villard, S., Ferrieres, G., Piechaczyk, M., Cerutti, M., Devauchelle, G. and Pau, B. (1999) FEBS Lett. 452, 386-394.

[47] Poul, M. A., Cerutti, M., Chaabihi, H., Devauchelle, G., Kaczorek, M. and Lefranc, M. P. (1995) Immunotechnology 1, 189-196.

[48] Summers, M. and Smith G. (1987) Texas Agricultural Experiment, Station Bulletin, 1555.

[49] Kappler, J., Kotzin, B., Herron, L., Gelfand, E. W., Bigler, R. D., Boylston, A., Carrel, S., Posnett, D. N., Choi, Y. and Marrack, P. (1989) Science 244, 811-813.

[50] Tillman, D. M., Jou, N. T., Hill, R. J. and Marion, T. N. (1992) J. Exp. Med. 176, 761-779.

[51] Sheehan, K. M., Mainville, C. A., Willert, S. and Brodeur, P.H. (1993) J. Immunol. 151, 5364-5375.

[52] Reinke, P., Volk, H. D., Miller, H., Neuhaus, K., Fietze, E., Herberger, J., Herberger, D., von Baehr, R. and Emmrich, F. (1991) Lancet 338, 702-703.

[53] Morel, P., Vincent, C., Cordier, G., Panaye, G., Carosella, E. and Revillard, J. P. (1990) Clin, Immunol. Immunopathol. 56, 311-322.

[54] Emiliani, S., Coudronnière, N., Delsert, C. and Devaux, C. (1996) J. Biomed. Sci. 3, 31-40.

[55] Sattentau, Q. J. and Weiss, R. A. (1988) Cell 52, 631-633.

[56] Arthos, J., Deen, K. C., Chaikin, M. A., Fornwald, J. A., Sathe, G., Sattentau, Q. J., Clapham, P. R., Weiss, R. A., McDougal, J. S. and Pietropaolo, C. (1989) Cell 57, 469-481.

[57] Mazerolles, F. and Fischer, A. (1998) Int. Immunol. 10, 1897-1905.

[58] Jauliac, S., Mazerolles, F., Jabado, N., Pallier, A., Bernard, F., Peake, J., Fischer, A. and Hivroz, C. (1998) Eur. J. Immunol. 28, 3183-3191.

[59] Jameson, B. A., McDonnell, J. M., Marini, J. C. and Korngold, R. (1994) Nature 368, 744-746.

[60] Dintzis, H. M., Symer, D. E., Dintzis, R. Z., Zawadzke, L. E. and Berg, J. M. (1993) Proteins 16, 306-308.

[61] Chothia, C., Gelfand, I. and Kister, A. (1998) J. Mol. Biol. 278, 457-479.

[62] Bhat, T. N., Bentley, G. A., Boulot, G., Greene, M. I., Tello, D., Dall'Acqua, W., Souchon, H., Schwarz, F. P., Mariuzza, R. A. and Poljak, R. J. (1994) Proc. Natl. Acad. Sci. USA 91, 1089-1093.

[63] Park, B. W., Zhang, H. T., Wu, C., Berezov, A., Zhang, X., Dua, R., Wang, Q., Kao, G., O'Rourke, D. M., Greene, M. I. and Murali, R. (2000) Nat. Biotechnol. 18, 194-198.

[64] Zhang, X., Gaubin, M., Briant, L., Srikantan, V., Murali, R., Saragovi, U., Weiner, D., Devaux, C., Autiero, M., Piatier-Tonneau, D. and Greene, M. I. (1997) Nat. Biotechnol. 15, 150-154.

[65] Murali, R. and Greene, M. I. (1998) Immunol. Res. 17, 163-169.

[66] Takasaki, W., Kajino, Y., Kajino, K., Murali, R. and Greene, M. I. (1997) Nat. Biotechnol. 15, 1266-1270.

[67] Foote, J. and Winter, G. (1992) J. Mol. Biol. 224, 48-499.

[68] Langedijk, J. P., Puijk, W. C., van Hoorn, W. P. and Meloen, R. H. (1993) J. Biol. Chem. 268, 16875-16878.

[69] Wu, H., Kwong, P. D. and Hendrickson, W. A. (1997) Nature 387, 527-530.

[70] Li, S., Satoh, T., Komgold, R. and Huang, Z. (1998) Immunol. Today 19, 455-462.

[71] Satoh, T., Aramini, J. M., Li, S., Friedman, T. M., Gao, J., Edling, A. E., Townsend, R., Koch, U., Choksi, S., Germann, M. W., Korngold, R. and Huang, Z. (1997) J. Biol. Chem. 272, 12175-12180.

[72] Huang, Z., Li, S. and Korngold, R. (1997) Biopolymers 43, 367-382.

[73] Kabat, E., Wu, T., Perry, H., Gottesman, K. and Foeller, C. (1991) Sequences of Proteins of Immunological Interest, 5th edn., US Department of Health and Human Services, Washington, D.C.

[74] P. J. Maddon, D. R. Littman, M. Godfrey, D. E. Maddon, L. Chess and R. Axel, The isolation and nucleotide sequence of a cDNA encoding the T cell surface protein T4; a new member of the immunoglobulin gene family, Cell 42 (1985), 93-104.

[75] J. H. Wang, Y. W. Yan, T. P. Garrett, J. H. Liu, D. W. Rodgers, R. L. Garlick, G. E. Tarr, Y. Husain, E. L. Reinherz and S. C. Harrison, Atomic structure of a fragment of human CD4 containing two immunoglobulin-like domains, Nature 348 (1990), 411-418.

[76] C. E. Rudd, J. M. Trevillyan, J. D. Dasgupta, L. L. Wong and S. F. Schlossman, The CD4 receptor is complexed in detergent lysates to a protein- tyrosine kinase (pp58) from human t lymphocytes. Proc Natl Acad Sci USA 85 (1988), 5190, 5194.

[77] R. Konig, L. Y. Huang and R. N. Germain, MHC class II interaction with CD4 mediated by a region analogous to the MHC class I binding site for CD8, Nature 356 (1992), 796-798.

[78] U. Dianzani, A. Shaw, B. K. Al-Ramadi, R. T. Kubo and C. A. Jr. Janeway, Physical association of CD4 with the T cell receptor, J Immunol 148 (1992), 678-688.

[79] A. Grakoui, S. K. Bromley, C. Sumen, M. M. Davis, A. S. Shaw, P. M. Allen and M. L. Dustin. The immunological synapse: a molecular machine controlling T cell activation. Science 285 (1999), 221-227.

[80] L. Briant and C. Devaux, Bioactive CD4 ligands and pre-and/or post-binding inhibitors of HIV-1, Adv Parmacol 48 (2000), 373-407.

[81] B. Huang, a. Yachou, S. Fleury, W. A. Hendrickson and R. P. Sekaly, Analysis of the contact sites on the CD4 molecule with class II MHC molecule: co-ligand versus co-receptor function, J Immunol 158 (1997), 216-225.

[82] P. Corbeau, M. Benkirane, R. Weil, C. David, S. Emiliani, D. Olive, C. Mawas, A. Serre and C. Devaux, Ig CDR3-like region of the CD4 molecule is involved in HIV-induced syncytia formation but not in viral entry, J Immunol 150 (1993), 290-301.

[83] L. Briant, N. Signoret, M. Gaubin, V. Robert-Hebmann, x. Zhang, R. Murali, M. I. Greene, D. Piatier-Tonneau and C. Devaux, Transduction of activation signal that follows HIV-1 binding to CD4 and CD4 dimerization involves the immunoglobulin CDR3-like region in domain 1 of CD4, J Biol Chem 272 (1997), 19441-19450.

[84] A. Truneh, D. Buck, D. R. Cassatt, R. Juszczak, S. Kassis, S. E. Ryu, D. Healey, R. Sweet and Q. Sattentau, A region in domain 1 of CD4 distinct from the primary gp120 binding site is involve din HIV infection and virus-mediated fusion, J Biol Chem 266 (1991), 5942-5948.

[85] M. Benkirane, P. Corbeau, V. Housset and C. Devaux, An antibody that binds the immunoglobulin CDR3-like region of the CD4 molecule inhibits provirus transcription in HIV-infected T cells, Embo 12 (1993), 4909-4921.

[86] M. Benkirane, H. Schmid-Antomarchi, D. R. Littman, M. Him, B. Rossi and C. Devaux, The cytoplasmic tail of CD4 is required for inhibition of human immunodeficiency virus type I replication by antibodies that bind to the immunoglobulin CDR3-like region in domain 1 of CD4, J Virol 69 (1995), 6904-6910.

[87] C. Dhiver, D. Olive, S. Rousseau, C. Tamalet, m. Lopez, J. R. Galindo, M. Mourens, M. Hirn, J. A. Gastaut and C. Mawas, Pilot phase I study using zidovudine in association with a 10-day course of anti-CD4 monoclonal antibody in seven AIDS patients, Aids 3 (1989), 835-842.

[88] P. M. Deckert, M. Ballmaier, S. Lang, H. Deicher and I. Schedel, CD4-imitating human antibodies in HIV infection and anti-idiotypic vaccination, J Immunol 156 (1996), 826-833.

[89] I. Schedel, G. C. Sutor, G. Hunsmann and E. Jurkiewicz, Phase II study of anti-CD4 idiotype vaccination in HIV positive volunteers, Vaccine 17 (1999), 1837-1845.

[90] M. Bruggemann, G. Winter, H. Waldmann and M. S. Neuberger, The immunogenicity of chimeric molecules, J Exp Med 170 (1989), 2153-2157.

[91] J. E. Looney, D. M. Knight, M. Arevalo-Moore, H. Trinh, K. I. Pak, M. R. Dalesandro, E. P. Rieber, G. Riethmuller, P. E. Daddona and J. Ghrabyeb, High-level expression and characterization of a mouse-human chimeric CD4 antibody with therapeutic potential, Hum Antibodies Hybridomas 3 (1992), 191-200.

[92] V. L. Pulito, V. A. Roberts, J. R. Adair, A. L. Rothermel, A. M. Collins, S. S. Varga, C. Martocello, M. Bodmer, L. K. Jollife and R. A. Zivin, Humanization and molecular modeling of the anti-CD4 monoclonal antibody, OKT4A, J Immunol 156 (1996), 2840-2850.

[93] R. Verma, E. Boleti and A. J. T. George, Antibody engineering: Comparison of bacterial, yeast, insect and mammalian expression systems, J Immunol Methods 216 (1998), 165-181.

[94] M. A. Poul, M. Cerutti, H. Chaabihi, G. Devauchelle, M. Kaczorek and M. P. Lefranc. Design of cassette baculovirus vectors for the production of therapeutic antibodies in insect cells, Immunotechnology 1 (1995), 189-196.

[95] M. A. Poul, M. Cerutti, H. Chaabihi, M. Ticchioni, F. A. Deramoudt, A. Bernard, G. Devauchelle, M. Kaczorek and M. P. Lefranc, Cassette baculovirus vectors for the production of chimeric, humanized, or human antibodies in insect cells, Eur J Immunol 25 (1995), 2005-2009.

[96] P. J. Maddon, S. M. Molineaux, D. E. Maddon, K. A. Zimmerman, M. Godfrey, F. W. Alt, L. Chess and r. Axel, Structure and expression of the human and mouse T4 genes, Proc Natl Acad Sci USA 84 (1987), 9155-9159.

[97] F. Manca, P. DeBerardinis, D. Fenoglio, M. N. Ombra, G. LiPira, D. Saverino, M. Autiero, L. Lozzi, L. Bracci and J. Guardiola, Antigenicity of HIV-derived T helper determinants in the context of carrier recombinant proteins: effect on T helper cell repertoire selection, Eur J Immunol 26 (1996), 2461-2469.

[98] L. Briant, V. Robert-Hebmann, C. Acquaviva, A. Pelchen-Matthews, M. Marsh and C. Devaux, the protein tyrosine kinase p561ck is required for triggering NF-kappaB activation upon interaction of human immunodeficiency virus type 1 envelope glycoprotein gp120 with cell surface CD4, J Virol 72 (1998), 6207-6214.

[99] T. Chardes, S. Villard, G. Ferrieres, M. Piechaczyk, M. Cerutti, G. Devauchelle and B. Paul, Efficient amplification and direct sequencing of mouse variable regions from any immunoglobulin gene family, FEBS Lett 452 (1999), 386-394.

[100] U. K. Laemmli, Cleavage of structural proteins during the assembly of the had of bacteriophage T4, Nature 227 (1970), 680-686.

[101] E. K. Molina, D. Laune, C. Gougat, B. Pau and C. Granier, Improved performances of spot multiple peptide synthesis, Pept Res 9 (1996), 151-155.

[102] R. Karisson, H. Roos, L. Fagerstam and B. Persson, Methods (Orlando) 6 (1994), 99-100.

[103] P. Morel, C. Vincent, G. Cordier, G. Panaye, E. Carosella and J. P. Revillard, Anti-CD4 monoclonal antibody administration in renal transplanted patients, Clin Immunol Immunopathol 56 (1990), 311-322.

[104] Q. J. Sattentau, J. Arthos, K. Deen, H. Hanna, D. Healey, P. C. Beverley, R. Sweet and A. Truneh, Structural analysis of the human immunodeficiency virus-binding domain of CD4. Epitope mapping with site-directed mutants and anti-idiotypes, J Exp Med 170 (1989),1319-1334.

[105] L. Briant, V. Robert-Hebmann, V. Sivan, A. Brunet, J. Pouyssegur and C. Devaux, Involvement of extracellular signal-regulated kinase module in HIV-mediated CD4 signals controlling activation of nuclear factor-kappa B and AP-1 transcription factors, J Immunol 160 (1998), 1875-1885.

[106] G. C. Sutor, U. Dreikhausen, U. Vaehning, E. Jurkiewicz, G. Hunsmann, K. Lundinand I, Schedel, Neutralization of HIV-1 by antio-idiotypes to monoclonal anti-CD4: potential for idiotype immunization against HIV, J Immunol 149 (1992), 1452-1461.

[107] E. H. Choy, D. J. Connolly, N. Rapson, S. Jeal, J. C. Brown, G. H. Kingsley, G. S. Panayi and J. M. Johnstone, Pharmacokinetic, pharmacodynamic and clinical effects of a humanized IgG1 anti-CD4 monoclonal antibody in the peripheral blood and synovial fluid of rheumatoid arthritis patients, Rheumatology 39 (2000), 1139-1146.

[108] A. B. Gottlieb, M. Lebwohl, S. Shirin, A. Sherr, P. Gilleaudeau, C. Songer, G. Solodkina, R. Grossman, E. Gisoldi, S. Phillips, H. M. Neisler and J. G. Krueger, Anti-CD4 monoclonal antibody treatment of moderate to severe psoriasis vulgaris: results of a pilot, multicenter, multiple-dose, placebo-controlled study, J Am Acad Dermatol 43 (2000), 595-604.

[109] E. Lopez, E. Racadot, M. Bataillard, E. Berger and L. Rumbach, Interferon gamma, IL2, IL4, IL10 and TNFalpha secretions in multiple sclerosis patients treated with an anti-CD4 monoclonal antibody, Autoimmunity 29 (1999), 87-92.

[110] B. W. Van Oosten, M. Lai, F. Barkhof, D. H. Miller, I. F. Moseley, A. J. Thompson, S. Hodgkinson and C. H. Polman, A phase II trial of anti-CD4 antibodies in the treatment of multiple sclerosis, Mult Scler 1 (1996), 339-342.

[111] F. L. Delmonico and A. B. Cosimi, Anti-CD4 monoclonal therapy, Clin Transplant 10 (1996), 397-403.

[112] D. A. Hafer, J. Ritz, S. Schlossman and H. Weiner, Anti-CD4 and anti-CD2 monoclonal antibody infusions in subjects with multiple sclerosis: immunosuppressive effects and human anti-mouse responses, J Immunol 141 (1988), 131-138.

[113] D. M. Fishwild, S. L. O'Donnell, t. Bengoechea, D. V. Hudson, F. Harding, S. L. Bernhard, D. Jones, R. M. Kay, K. M. Higgins, S. R. Schramm and N. Lonberg, High-avidity human IgG kappa monoclonal antibodies from a novel strain of minilocus transgenic mice, Nat Biotechnol 14 (1996), 845-851.

[114] D. Anderson, K. Chambers, N. Hanna, J. Leonard, M. Reff, R. Newman, J. Baldoni, D. Dunleavy, M. Reddy, R. Sweet and A. Truneh, A primatized-Mab to human CD4 causes receptor modulation, without marked reduction in DC4⁺ T cells in chimpanzees; in vitro and in vivo characterization of a Mab (IDEC-CE91) to human CD4, Clin Immunol Immunopathol 84 (1997), 73-84.

[115] O. M. Kon, B. S. Sihra, C. H. Compton, T. B. Leonard, A. B. Kay and N. C. Barnes, Randomized, dose-ranging, placebo-controlled study of chimeric antibody to CD4 (keliximab) in chronic severe asthma. Lancet 352 (1998), 1109-1113.

[116] P. A. Van der Lubbe, C. Reiter, F. C. Breedveld, K. Druger, M. Schattenkirchner, M. E. Sanders and G. Riethmuller, Chimeric CD4 monoclonal antibody cM-T412 as a therapeutic approach to rheumatoid arthritis, Arthritis Rheumatism 36 (1993), 1375-1379.

[117] P. Hutchings, L. O'Reilly, N. M. Parish, H. Waldman and A. Cooke, The use of non-depleting anti-CD4 monoclonal antibody to reestablish tolerance to cells in NOD mice, Eur J Immunol 22 (1992), 1913-1918.

[118] C. A. Haseman and D. Capra, High-level production of a functional immunoglobulin heterodimer in a baculovirus expression system, Proc natl acad sci USA 87 (1990), 3942-3946.

[119] K. N. Potter, Y. Li and D. Capra, Antibody production in the baculovirus expression system, Int Rev Immunol 10 (1993), 103-112.

[120] W. J. Ernst, A. Spenger, L. Toellner, H. Katinger and R. M. Grabher, Expanding baculovirus surface display, Eur J Biochem 267 (2000), 4033-4039.

[121] Y. Boublik, P. DiBonito and I. M. Jones, Eukaryotic virus display: engineering the major surface glycoprotein of Autographa californica nuclear polyhedrosis virus (AcNPV) for the presentation of foreign proteins on the cell surface, Biotechnology 13 (1995), 1079-1084.

[122] M. Liang, S. Dübel, D. Li, I. Queitsch, W. Li and E. K. F. Bautz, Baculovirus expression cassette vectors for rapid production of complete human IgG from phage display selected antibody fragments, J Immunol Methods 247 (2001), 119-130.

[123] C. Lemeulle, t. Chardès, C. Montavon, H. Chaabihi, J. C. Mani, M. Pugnière, M. Cerutti, G.

Devauchelle, . Pau and M. Biard-Piechaczyk, Anti-digoxin scFv fragments expressed in bacteria and in insect cells have different antigen binding properties, FEBS Lett 423 (1998), 159-166.

[124] K. M. Lindley, J. L. Su, P. K. Hodges, G. B. Wisely, R. K. Bledsoe, J. P. Condreay, D. A. Winegar, J. T. Hutchings and T. A. Kost, Production of monoclonal antibodies using recombinant baculovirus displaying gp-64 fusion proteins, J Immunol Methods 234 (1999), 123-135.

[125] E. Li, S. L. Brown, C. S. Dolman, G. B. Brown and G. R. Nemerow, Production of functional antibodies generated in a nonlytic insect cell expression system, Protein Expr Puriƒ21 (2001), 121-128.

What is claimed is:

1. An isolated CDR-derived polypeptide from monoclonal antibody 13B8.2.

2. A polypeptide according to claim 1 having a CDR domain which is a CDR-H domain.

3. A polypeptide according to claim 1 having a CDR domain which is a CDR-L domain.

4. An isolated polypeptide according to claim 1 comprising a sequence selected from the group consisting of:

| | |
|---|---|
| LTTFGVHWVRQS: | SEQ ID No.1 |
| TTFGVHWVRQSP: | SEQ ID No.2 |
| GVIWRSGITDYN: | SEQ ID No.3 |
| WRSGITDYNVPF: | SEQ ID No.4 |
| YYCAKNDPGTGF and: | SEQ ID No.5 |
| KNDPGTGFAYWG.: | SEQ ID No.6 |

5. An isolated polypeptide according to claim 1 comprising a sequence selected from the group consisting of:

| | | |
|---|---|---|
| TCRASENIYSYL | | SEQ ID No.7 |
| IYSYLAWYQQKQ and: | SEQ ID No.8 | |
| YYCQHHYGNPPT.: | SEQ ID No.9 | |

6. An isolated polypeptide according to claim 3 comprising a sequence selected from the group consisting of:

| | |
|---|---|
| TCRASENIYSYL: | SEQ ID No.7 |
| IYSYLAWYQQKQ and: | SEQ ID No.8 |
| YYCQHHYGNPPT.: | SEQ ID No.9 |

7. A pharmaceutical composition comprising a pharmaceutically acceptable carrier and at least a peptide according to claims 1 to 6.

8. A method for inhibiting an immune response in a patient, comprising administering to the patient a therapeutically effective amount of a pharmaceutical composition according to claim 7.

9. A method for treating a subject suffering from an autoimmune disorder, comprising administering to said subject a therapeutically effective amount of a pharmaceutical composition according to claim 7.

10. The method according to claim 9, wherein said autoimmune disorder is selected from the group consisting of psoriasis, rhumatoid arthritis and lupus erythematosus.

11. A method for treating a subject suffering from a transplant rejection comprising administering to said subject a therapeutically effective amount of a pharmaceutical composition according to claim 7.

12. A method for treating a subject suffering from an HIV immunodeficiency disorder comprising administering to said subject a therapeutically effective amount of a pharmaceutical composition according to claim 7.

13. A method for treating a subject suffering from a tumoral disorder comprising administering to said subject a therapeutically effective amount of a pharmaceutical composition according to claim 7.
